(19) United States
(12) Patent Application Publication
(10) Pub. No.: US 2014/0037656 A1
(43) Pub. Date: Feb. 6, 2014

(54) Treatment for Tauopathies

(71) Applicant: Mitchell S. Felder, Hermitage, PA (US)

(72) Inventor: Mitchell S. Felder, Hermitage, PA (US)

(21) Appl. No.: 14/110,994

(22) PCT Filed: Apr. 25, 2012
(86) PCT No.: PCT/US12/35021

§ 371 Date: Oct. 10, 2013

Related U.S. Application Data
(60) Provisional application No. 61/488,741, filed on May 22, 2011.
(60) Provisional application No. 61/481,164, filed on Apr. 30, 2011.

(51) Int. Cl. A61K 35/30; A61M 1/00

(52) U.S. Cl. CPC A61K 35/30; A61M 1/00
USPC 424/178.1; 424/537

(57) ABSTRACT

A method is described to treat tauopathies by extracorporeally treating a patient's cerebrospinal fluid (CSF). The method includes introducing an antibody into the CSF that is targeted to an antigen associated with tauopathies. The antibody can include an albumin moiety, and targets the removal of antigens such as tau protein, phosphorylated tau (pTau) protein, Ubiquitin and PKN. The antibody-antigen complex can be removed from the CSF and the CSF can be returned to the patient.

### FIELD OF THE INVENTION

The present invention relates to a treatment for tauopathies, that is, aggregations of tau proteins which form neurofibrillary tangles in the central nervous system.

### BACKGROUND OF THE INVENTION

Tauopathies are class of neurodegeneration in which an aggregation of tau proteins forms neurofibrillary tangles in the brain. Examples of Tauopathies include Alzheimer's disease, pseudobulbar palsy, frontotemporal lobe degeneration—also known as Picks disease, progressive supranuclear palsy, frontotemporal dementia, traumatic brain injury (TBI), chronic traumatic encephalopathy (CTE), and corticobasal degeneration

Neuropathology has shown close similarities between the neuropathology of Alzheimer's disease, frontotemporal dementia, TBI and CTE. All show abnormal neurofibrillary depositions in the brain. For example, Alzheimer's disease, TBI and CTE exhibit a triad of symptomatology: consisting of cognitive impairment, irrational and impulsive behavior, and depression. This symptomatology has been found in some measure in all tauopathies.

Presently more than 5 million Americans are afflicted with Alzheimer's disease. By the year 2058 this number will increase to approximately 13.4 million Americans. Similarly, more than two million patients suffer annually from TBI or CTE, including 52,000 deaths and 275,000 hospitalizations in the United States each year. A head injury occurs in the civilian population of United States every 7 seconds. The peak morbidity and mortality from TBI and CTE occurs in patients between the ages of 15-24. Men are affected four times as often as women.

Before the age of 55 the prevalence of patients with Alzheimer's disease is less than one percent, but this increases to 10% percent by the age of 65. The percentage is as high as 40% at age 85 and older. The number of new cases arising over a specific period of time rises steeply from less than 1% of the population before the age of 65 to 6% per year for individuals age 85 and older. These patients suffer a great deal of morbidity during the course of this illness with the average duration of morbidity until death ranging from 4 to 16 years. Estimates place the total cost for treating patients with tauopathies in the United States at over 630 billion dollars over the next twenty years.

No treatments are currently available that are able to halt or reverse the neuropathologic findings in tauopathies. A treatment for stopping and possibly reversing the progression of such neuropathologies would be extremely beneficial.

### SUMMARY OF THE INVENTION

The present invention relates to an article and method of extracorporeal treating a patient's cerebrospinal fluid (CSF). U.S. 61/160,755and U.S. 61/148,431 are hereby incorporated by reference. The treatment includes a plurality of stages comprising removing CSF from a patient, applying an extracorporeal treatment to the CSF, and returning the CSF to the patient.

In the first stage of the treatment, the CSF is removed from the patient. A convenient method for removing CSF includes a standard lumbar puncture. In the second stage, a treatment is applied to the CSF. The treatment can include an antibody directed against a targeted antigen. The third stage comprises returning the CSF to the patient and can also include removing the treatment from the CSF.

Antibodies can be produced that attack targeted antigens correlated with tauopathies, including Tau protein, phosphorylated Tau protein (pTau), Ubiquitin and PKN (a 120 kDa lipid-activated serine/threonine kinase).

### DETAILED DESCRIPTION OF THE INVENTION

The method of the present invention comprises treating a patient's CSF extracorporeally with an antibody designed to react with a targeted antigen. The antibody can include a moiety, for example, an albumin moiety, that can complex with the targeted antigen and permit efficacious dialysis of the antibody-antigen complex. Dialysis methods are well known by one skilled in the art.

In an embodiment of the invention, the antibody comprises an albumin moiety and targets the removal of tau protein, phosphorylated tau (pTau) protein, Ubiquitin and PKN. pTau protein can include tau phosphorylated with multiple epitopes (antigenic determinants), threonine 181+231, threonine 181, threonine 231+serine 235, serine 199, threonine 231, and serine 396+404.

The pTau protein in any CSF can be differentiated utilizing standard ELISA methodology. ELISA (enzyme-linked immunosorbant assay) is a biochemical technique which allows for the detection of an antigen in a sample. In ELISA, an antigen is affixed to a surface, and then an antibody binds to the antigen. The antibody is linked to an enzyme which enables a color change in the substrate.

In alternative embodiments, the targeted antibody can include a designer antibody with an attached macromolecular moiety. The antibody-macromolecular moiety complex will have a molecular size, and the macromolecular moiety is conveniently 1.000 mm to 0.005 mm in diameter. At least one microscreen can block the antibody-macromolecular moiety-targeted antigen complex from passing into the patient, that is, the CSF or other body fluid. The microscreen can include a series of microscreens. The microscreens can define openings having diameters sufficient to block passage of the complex. Conveniently, the diameters can be less than 50% of the diameter of the designer antibody-macromolecular moiety complex. The microscreen opening(s) should have a diameter of at least 25 micrometers in order to permit passage and return to circulation of the nonpathologic constituents.

In other embodiments, antibody microarrays capture an antibody-antigen complex comprising the targeted antigen. The antibody microarrays comprise a plurality of monoclonal antibodies attached to a surface. The microarrays can include millions of such monoclonal antibodies. After sufficient extracorporeal exposure of the complex to the antibody microarrays, the microarrays can become saturated or nearly saturated with the complex, and the microarrays can be replaced.

Still another embodiment of the present intervention comprises removing a targeted antigen from a bodily fluid using a designer antibody containing an iron (Fe) moiety. The combination of targeted antigen, antibody and iron moiety comprise an Fe-Antibody-Antigen complex. This complex can then be efficaciously removed using a strong, localized magnetic force field.

The invention comprises at least three stages including a first stage, a second stage and a third stage. The first stage comprises removing CSF from a patient. Removal can occur using any convenient method including, for example, a spinal tap. The second stage treats the CSF. The thirds stage returns the treated CSF to the patient.

The treatment can include the removal of the tau protein, pTau, Ubiquitin and/or PKN from the CSF. The cleansed CSF can then be returned to the patient, such as, for example by using the same catheter that was originally used in removing the CSF. In one embodiment, the treatment of CSF comprises removing 5-25 ml of CSF from a patient, and applying the treatment to CSF before returning it to the patient. The frequency of such treatments would depend upon the underlying symptomatology and pathology of the patient.

The article of the invention includes two-stages. The first stage includes an inlet for CSF and at least one exterior wall defining a treatment chamber that is fluidly connected to a second stage. The second stage comprises a removal module and an outlet for the CSF. In embodiments, the removal module is selected is selected from a group comprising a mechanical filter, a chemical filter, a dialysis machine, a molecular filter, molecular adsorbant recirculating system (MARS), a plasmapharesis unit, or combinations thereof.

The method includes removing CSF from a patient in a first stage, treating the CSF and optionally removing the treatment from the CSF in a second stage, and returning the CSF to the patient in a third stage. The CSF can be removed from the patient using any convenient method, including standard lumbar puncture procedure. The second stage can include sequentially passing the extracorporeal bodily fluid through a treatment chamber and a removal module.

The second stage applies a treatment to the CSF, which can include introducing a designer antibody that joins with an antigen in the CSF to form an antibody-antigen complex. The antibody-antigen complex can be removal the CSF in the removal module. Optionally, the antibody-antigen complex can be conjugated with a second antibody comprising a moiety that increases the efficacy of removal to form an antibody-moiety-antigen complex.

In the third stage, the purified CSF (CSF with removed targeted antigens, Tau, pTau, Ubiquitin and/or PKN) is then returned to the patient.

The device of the invention comprises a first stage including an inlet for CSF and at least one exterior wall defining a treatment chamber that is fluidly connected to a second stage comprising a removal module and an outlet for the CSF. The treatment chamber can include a delivery tube for introducing a treatment into the treatment chamber. In embodiments, the delivery tube comprises a hollow tube including at least one interior wall defining a plurality of holes through which the treatment can be added to the treatment chamber. The treatment can also be delivered through the hollow tube in counter-current mode with reference to the flow of the extracorporeal CSF. The removal module can be any device capable of removing the antibody-antigen complex. In embodiments, the removal module is selected from a group comprising a mechanical filter, a chemical filter, a dialysis machine, a molecular filter, molecular adsorbant recirculating system (MARS), a plasmapharesis unit, or combinations thereof.

In an example, the first stage of the device applies a treatment of an antibody with an attached albumin moiety that targets the antigens Tau, pTau, Ubiquitin and/or PKN. The second stage includes substantial removal of the treatment from the extracorporeal CSF bodily fluid.

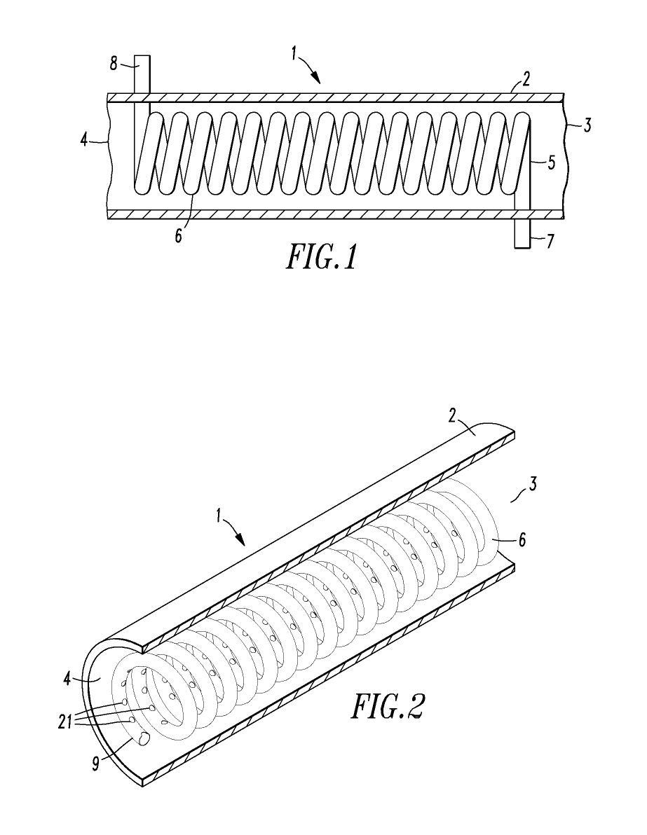

As shown in FIG. 1, the first stage can include an exterior wall 2 defining a treatment chamber 5. The treatment can be applied in the treatment chamber 5. Residence times of the CSF can be altered by changing the dimensions of the treatment chamber or the flow rate of the CSF through the treatment chamber 5. CSF fluid enters the inlet 3, passes through the treatment chamber 5, and exits the outlet 4. In embodiments, the treatment can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the CSF. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer antibodies in the CSF. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic.

The antibody with attached albumin moiety, targeting the antigens Tau, pTau, Ubiquitin, and PKN can be delivered in a concurrent or counter-current mode with reference to the CSF. In counter-current mode, the CSF enters the treatment chamber 5 at the inlet 3. The designer antibody can enter through a first lead 8 near the outlet 4 of the treatment chamber 5. CSF then passes to the outlet 4 and the designer antibodies pass to the second lead 7 near the inlet 3. The removal module of the second stage substantially removes the designer antibodies-antigen molecular compound from the CSF.

The second stage can include a filter, such as a dialysis machine, which is known to one skilled in the art. The second stage can include a molecular filter. For example, molecular adsorbants recirculating system (MARS), which may be compatible and/or synergistic with dialysis equipment. MARS technology can be used to remove small to average sized molecules from the CSF. Artificial liver filtration presently uses this technique.

The methodology can include a plurality of steps for removing the targeted antigens (Tau, pTau, Ubiquitin and PKN). A first step can include directing a first antibody against the targeted antigen. A second step can include a second antibody. The second antibody can be conjugated with albumen, or alternatively a moiety which allows for efficacious dialysis. The second antibody or antibody-albumen complex combines with the first antibody forming an antibody-antibody-moiety complex. A third step is then utilized to remove the complex from the CSF. This removal is enabled by utilizing dialysis and/or MARS. The purified CSF can then be returned to the patient.

In practice, a portion of the purified CSF can be tested to ensure a sufficient portion of the targeted antigens (Tau, pTau, ubiquitin and P KN) have been successfully removed from the CSF. Testing can determine the length of treatment and evaluate the efficacy of the sequential dialysis methodology in removing the targeted antigens. CSF with an unacceptably large concentrations of complex remaining can then be refiltered before returning the CSF to the patient.

In embodiments, the second stage to remove the antibody-moiety-targeted antigen complex by various techniques including, for example, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or diasylate flow rate can increase the rate of removal of the antibody with attached albumin moiety targeting the antigens Tau, pTau, ubiquitin and PKN.

Addition embodiments can include continuous renal replacement therapy (CRRT) which can remove large quantities of filterable molecules from the extracorporeal CSF. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis.

The sieving coefficient (SC) is the ratio of the molecular concentration in the filtrate to the incoming CSF. A SC close to zero implies that the moiety antibody-targeted antigen complex will not pass through the filter. A filtration rate of 50 ml per minute is generally satisfactory. Other methods of increasing the removability of the moiety-antibody-targeted antigen include the use of temporary acidification of the CSF utilizing organic acids to compete with protein binding sites.

Numerous modifications and variations of the present invention are possible. It is, therefore, to be understood that within the scope of the following claims, the invention may be practiced otherwise than as specifically described. While this invention has been described with respect to certain preferred embodiments, different variations, modifications, and additions to the invention will become evident to persons of ordinary skill in the art. All such modifications, variations, and additions are intended to be encompassed within the scope of this patent, which is limited only by the claims appended hereto.

1. A method for treating cerebrospinal fluid (CSF) characterized by:
a. removing CSF from a patient in a first stage;
b. applying a treatment to the CSF in a second stage; and
c. returning the CSF to the patient in a third stage.

2. The method of claim 1, further characterized by the second stage including removing the treatment from the CSF.

3. The method of claim 1, further characterized by the treatment including
a. introducing a targeted antibody that joins with an antigen in the CSF to form an antibody-antigen complex; and
b. removing the complex form the CSF.

4. The method of claim 1, further characterized by the treatment including
a. introducing a targeted antibody that joins with an antigen in the CSF to form an antibody-antigen complex; and
b. conjugating the antibody-antigen complex with a second antibody comprising a moiety that increases the efficacy of removal to form an antibody-moiety-antigen complex.

5. The method of claim 1, further characterized by determining efficacy of treatment by testing the CSF after the treatment and before returning the CSF to the patient.

6. The method of claim 3, further characterized by the targeted antibody comprising an albumin moiety.

7. The method of claim 3, further characterized by the antigen being selected from a group consisting of Tau protein, pTau protein, Ubiquitin and PKN.

8. The method of claim 7, further characterized the pTau protein being selected from a group consisting of tau phosphorylated with multiple epitopes (antigenic determinants), threonine 181+231, threonine 181, threonine 231+serine 235, serine 199, threonine 231, and serine 396+404.

9. The method of claim 7, further characterized by differentiating the pTau protein using an enzyme-linked immunosorbant assay.

10. The method of claim 3, further characterized by the targeted antibody comprising an antibody-macromolecular complex comprising the antibody attached to a macromolecular moiety, the antibody-macromolecular complex having a molecular size.

11. The method of claim 10, further characterized by the macromolecular moiety having a diameter from 0.005 mm to 1.000 mm.

12. The method of claim 10, further characterized by the treatment comprising a microscreen that blocks the antibody-macromolecular complex from returning to the patient.

13. The method of claim 12, further characterized by the treatment comprising a series of microscreens.

14. The method of claim 12, further characterized by the microscreen defining openings having diameters sufficient to block passage of the antibody-macromolecular complex.

15. The method of claim 14, further characterized by the diameters of the openings being at least 25 micrometers and less than 50% of the molecular size.

16. The method of claim 3, further characterized by the treatment comprising antibody microarrays able to capture the antibody-antigen complex.

17. The method of claim 17, further characterized by the antibody microarrays comprising a plurality of monoclonal antibodies attached to a surface.

18. The method of claim 3, further characterized by the treatment comprising forming an Iron-Antibody-Antigen complex by combining an iron moiety, the antibody and the antigen, and removing the Iron-Antibody-Antigen complex using a strong, localized magnetic force field.

19. The method of claim 1, further characterized by the treatment being selected from a group comprising a mechanical filter, a chemical filter, a dialysis machine, a molecular filter, molecular adsorbant recirculating system, a plasmapharesis unit, or combinations thereof.
